(19) United States
(12) Patent Application Publication
(10) Pub. No.: US 2009/0018484 A1
(43) Pub. Date: Jan. 15, 2009

(54) System device and method for oxygenation

(76) Inventor: Alexander B. Levitov, Roanoke, VA (US)

Correspondence Address: BECK AND TYSVER P.L.L.C., 2900 THOMAS AVENUE SOUTH, SUITE 100, MINNEAPOLIS, MN, 55416, US

(21) Appl. No.: 11/827,168

(22) Filed: Jul. 11, 2007

(51) Int. Cl. A61M 1/16

(52) U.S. Cl. 604/6.14

(57) ABSTRACT

A liquid to liquid gas exchanger for supporting the human respiration function.

### FIELD OF THE INVENTION

The present invention relates generally to methods and apparatus for providing oxygen to a patient's blood. More particularly the device and method incorporate the use of a portable gas exchange device and associated equipment that can supplement the oxygenation process performed by the lungs of the patient.

### BACKGROUND OF THE INVENTION

The traditional method of blood oxygenation is called ECMO for extracorporeal membrane oxygenation. In general such devices include a pump that extracts blood from the body, passes it through a membrane oxygenator and returns the blood to the patient's circulation. The presence of the oxygenator and the extracorporeal pump permit the system to perform as a heart/lung bypass and such devices are widely used to perform open heart or still heart surgery. Such devices have also been used to treat patients in respiratory failure, although such use is minimal.

In general the prior art device will include a permeable membrane with one side exposed to a gas containing oxygen and the other side exposed to the blood. Small pores within the membrane permit gas exchange between the blood and the gas side of the system effectively functioning as a “lung.”

The device and method of this invention can be used to treat the approximately 150,000 cases of respiratory failure that occur each year in the United States. Many of these cases are currently treated with positive pressure ventilation (PPV). Although PPV is useful it is difficult to control and patents may suffer Barotraumas, Sheertraumas, Volutrauma, Biotrauma, and ventilator associated pneumonia.

### SUMMARY OF THE INVENTION

By way of contrast the present invention relies on an oxygen rich carrier fluid to exchange gases including oxygen through a membrane. In some configuration the membrane separates the patients blood from the gas carrier fluid. The liquid to liquid oxygen transfer process uses a membrane to mediate the oxygen transfer process and resembles a “gill” rather than a “lung”.

### BRIEF DESCRIPTION OF DRAWINGS

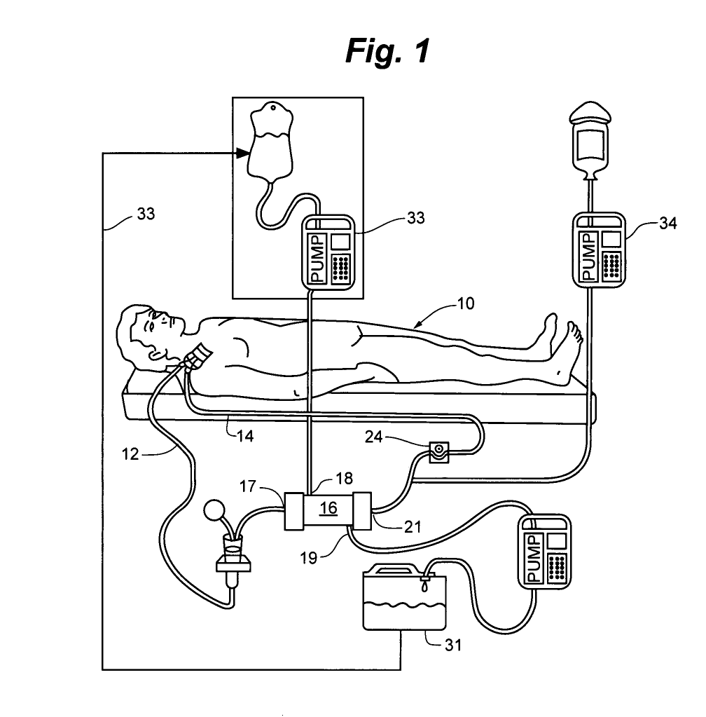

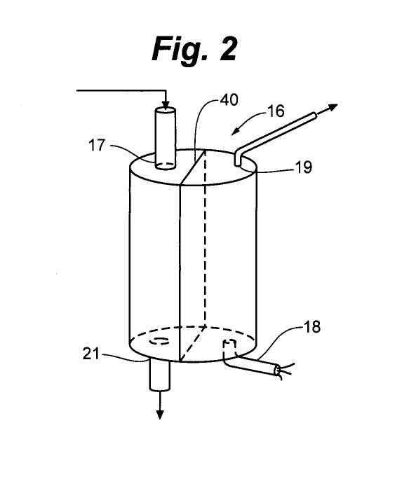

Throughout the several figures of the drawings identical reference numerals indicate equivalent structure wherein FIG. 1 is a system level schematic showing the various componetry and its relationship to the patient; and FIG. 2 is a schematic cross section of an oxygenator device.

### DETAILED DESCRIPTION OF THE PREFERRED EMBODIMENT

In the figure the patient 10 is cannulated on the venous side (V) through cannula 12, which extracts blood. Ultimately this blood is oxygenated and returned to the patient through cannula 14, also on the venous side. Although arterial to venous (A to V) cannulation is possible, it is preferred to operate V to V. The blood taken from the patient is passed through a oxygenator 16 chamber, which has a “blood side” inlet 17 and a fluid oxygen carrier side inlet 18. In operation oxygenated carrier fluid is passed through the membrane oxygenator from the inlet side 18 to the outlet 19 at a rate sufficient to increase the partial pressure of oxygen in the patient's blood. The device need not function to completely supplant the lungs for the patient but rather may be used to increase the amount of oxygen available for metabolism while the lungs are healing from a disease or acute injury. In the figure the pump 24 meters, regulates and controls the rate of blood flow through the system, however, it should be recognized that the pressure drop across the pump is quite modest and in fact it may be possible to drive blood through the system without a pump, relying solely upon the patient's cardiac output. Returning to the figure the oxygen carrier liquid may be pumped through the membrane oxygenator and discarded as shown in FIG. 1 by discard reservoir 31. Or in the alternative the fluid may be recycled as indicated by arrow 30 through return line 33. It should also be recognized that a CO₂ scrubber can be inserted into the deoxygenated fluid line 33 to maintain adequate control over blood pH and the like when the carrier fluid is recycled. With respect to oxygen carrier fluids a number of artificial blood materials are commercially available and are ideal for this acute application. The flow of the carrier fluid though the system may be regulated and motivated by the use of a computer controlled pump 33. To minimize coagulation a heparin solution may be infused through a computer controlled pump 34 on the low-pressure side of roller pump 24. Turning to FIG. 2 there is shown an oxygenator 16 shown in schematic cross section where venous blood from the patient enters through port 17 and exits through port 21. The blood is separated from the oxygen carrier in chamber 60 by a semi permeable membrane 40. The semi permeable membrane may have relatively large pore size because the carrier molecules and blood components are large. This permits a substantial reduction in the amount of membrane area exposed to the blood which is a benefit because it reduces hemolysis of the blood. For effective therapy the membrane oxygenator need not operate at a wide oxygen disparity, i.e. the partial pressure of oxygen on each side of the membrane may be close since the difference between suffocation and survival is a partial pressure of oxygen between 40 and 50 resulting in a 60 to 90 percent blood oxygen saturation. It has been shown that hemoglobin saturation curve is very steep between these numbers, which make the device practical in small sizes. Since the heart is functioning in the patient only a small fraction of the cardiac output on the order of 10 percent need be shunted into the system for hyper oxygenation. In this regard the degree of blood saturation of oxygen is substantially higher than in conventional membrane oxygenators.

### EXAMPLE

An experimental version of the device and method have been carried out using a dialysis filter as the gas exchanger. Hemopure was used as the oxygen carrier, which is a commercially available blood substitute product. A peristaltic pump pulls venous blood into the system and returns the blood into the venous system. The pre membrane blood pH was 6.4 while the post membrane pH was 7.016, the partial pressure of carbon dioxide pre membrane was pCO2 71 mm Hg and 16 mm Hg post membrane. The partial pressure of oxygen pO2 was 37 mm Hg and pre membrane and 250 mm Hg after the membrane. These figures show significant improvement over values of Co2 and pH over prior art techniques.

What is claimed is:

1. A method of supporting lung function comprising the steps of:
a. canulating the patient to remove blood from the body;
b. passing the blood though a chamber, the chamber having a semi porous membrane, separating a blood side from an oxygen carrier side;
c. passing a liquid oxygen carrier fluid though the chamber on the oxygen carrier side separated from the blood by the membrane;
whereby oxygen in the liquid oxygen carrier fluid passes though the membrane and carbon dioxide and other gases are passed into the oxygen carrier fluid.

2. The method of claim 1 wherein the oxygen carrier fluid is artificial blood.

3. The method of claim 1 wherein the passing step for blood is performed by a peristaltic roller pump.

4. The method of claim 1 wherein the passing step for oxygen carrier fluid is performed by a computer controlled pump.
